(19) United States
(12) Patent Application Publication
(10) Pub. No.: US 2017/0176097 A1
(43) Pub. Date: Jun. 22, 2017

(54) Systems and Methods for Recovering Desired Light Hydrocarbons from Refinery Waste Gas Using a Back-End Turboexpander

(71) Applicant: Bechtel Hydrocarbon Technology Solutions, Inc., Houston, TX (US)

(72) Inventors: Berj Garbouchian, Houston, TX (US); Sudhir Golikeri, Richmond, TX (US); Jie Yu, Katy, TX (US); Vincent Leung, Houston, TX (US)

(21) Appl. No.: 15/180,291

(22) Filed: Jun. 13, 2016

Related U.S. Application Data
(63) Continuation of application No. 15/103,760.

(51) Int. Cl. F25J 3/02; C07C 7/11; C07C 7/04

(52) U.S. Cl. CPC F25J 3/0219; C07C 7/04; C07C 7/11; F25J 3/0238; F25J 3/0233; F25J 2200/06; F25J 2210/12; F25J 2200/72; F25J 2220/02; F25J 2220/66; F25J 2240/02; F25J 2270/12; F25J 2205/02; F25J 2280/40; F25J 2200/40

(57) ABSTRACT

Systems and methods for recovering light hydrocarbons from refinery waste gas using a back-end turboexpander to generate a higher recovery of the light hydrocarbons for use as petrochemical feedstock and to remove the liquid light hydrocarbons before entering the turboexpander.

### FIELD

This application is a continuation of U.S. patent application Ser. No. 15/103,760, which is incorporated herein by reference, and claims the priority of PCT Patent Application Serial No. PCT/US15/66668, filed on Dec. 18, 2015, which is incorporated herein by reference.

The present disclosure generally relates to systems and methods for recovering desired light hydrocarbons from refinery waste gas using a back-end turboexpander. More particularly, the present disclosure relates to recovering desired light hydrocarbons from refinery waste gas using a back-end turboexpander to generate a higher recovery of the light hydrocarbons for use as petrochemical feedstock and to remove the heavier hydrocarbons before entering the turboexpander.

### BACKGROUND

Gas from the streams in industrial applications, particularly hydrocarbon refining operations, often include methane, other constituents, and light hydrocarbons having a molecular weight equal to or greater than ethylene, including ethylene, ethane, propylene, propane, butylenes and butane (hereinafter collectively referred to as the “desired light hydrocarbons”). The desired light hydrocarbons therefore, comprise. Recovery of the desired light hydrocarbons is preferred because the desired light hydrocarbons are more valuable as petrochemical feedstock than as refinery fuel gas. However, the systems and methods for recovery of the desired light hydrocarbons are limited.

Current recovery of the desired light hydrocarbons in different refinery units such as a saturated gas plant, a coker gas plant and a fluid catalytic cracker (FCC) gas plant (collectively referred to as the “refinery gas plants”) is accomplished using absorption-stripping. The recovery of propane using absorption-stripping is in the 90-94% range while ethane is usually not recovered.

More recently, some of the desired light hydrocarbons have been recovered from the refinery waste gas using cryogenic systems. The general configuration of these cryogenic systems consists of first compressing, cooling and drying the feed gas to obtain a treated gas, followed by processing the treated gas through a turboexpander to produce a two phase result. The lowest temperatures of these cryogenic systems are reached in the turboexpander. The liquid generated from the treated gas passing through the turboexpander is separated from the vapor and sent to a distillation column that separates the desired light hydrocarbons from methane and other light components. The column overhead vapor and the turboexpander vapor are used as refinery fuel gas. Recovery of the ethane with this method is typically not more than 80%.

Conventional cryogenic systems have shortcomings. The presence of heavy hydrocarbons in the treated gas that can result in undesirable freezing within the turboexpander, frustrating operation of the cryogenic system. Additionally, the efficiency of recovery of the desired light hydrocarbons in convention systems is limited.

### SUMMARY

The present disclosure overcomes one or more deficiencies in the prior art by providing systems and methods for recovering the desired light hydrocarbons from refinery waste gas using a back-end turboexpander to generate a higher recovery of the desired light hydrocarbons for use as petrochemical feedstock and to reduce the extent of the heavy hydrocarbons entering the turboexpander.

In one embodiment, the present disclosure includes a system for recovery of light hydrocarbons, which comprises: i) gas chiller/dryer; ii) a distillation column connected to the gas chiller/dryer for recovering the light hydrocarbons; iii) a first liquid/gas separator connected to the distillation column for separating the light hydrocarbons in a liquid state from the distillation column and the light hydrocarbons in a gas state from the distillation column; and iv) a turboexpander connected to the first liquid/gas separator for cooling the light hydrocarbons in a gas state to produce a condensed liquid and a remaining vapor.

In another embodiment, the present disclosure includes a method for recovering light hydrocarbons from a gas stream, which comprises: i) treating the gas stream by compressing, amine-treating, drying and chilling to produce a residue lighter gas; and ii) separating the residue lighter gas in a distillation column between an overhead product and a raw column bottom liquid product containing the light hydrocarbons.

Additional aspects, advantages and embodiments of the disclosure will become apparent to those skilled in the art from the following description of the various embodiments and related drawings.

### BRIEF DESCRIPTION OF THE DRAWINGS

The present disclosure is described below with references to the accompanying drawings, in which like elements are referenced with like numerals, wherein:

FIG. 1 is a schematic diagram illustrating a system for recovering light hydrocarbons from refinery waste gas using a back-end turboexpander.

FIG. 2 is a schematic diagram illustrating another system for recovering light hydrocarbons from refinery waste gas using a back-end turboexpander and a heat exchanger.

### DETAILED DESCRIPTION

The subject matter of the present disclosures is described with specificity; however, the description itself is not intended to limit the scope of the disclosure. The subject matter thus, might also be embodied in other ways, to include different steps or combinations of steps similar to the ones described herein, in conjunction with other present or future technologies. Moreover, although the term “step” may be used herein to describe different elements of methods employed, the term should not be interpreted as implying any particular order among or between various steps herein disclosed unless otherwise expressly limited by the description to a particular order. While the following description refers refinery gas plants, the systems and methods of the present disclosure are not limited thereto and may be applied in other refineries to achieve similar results.

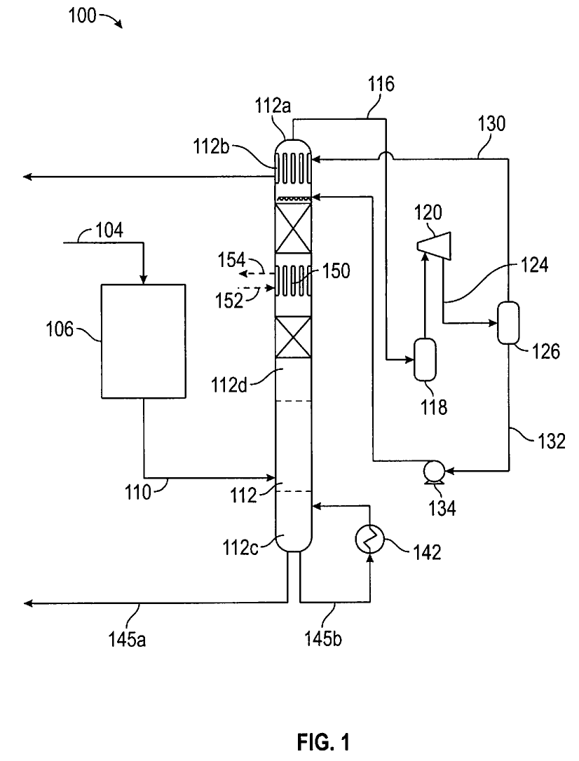

Referring now to FIG. 1, a schematic diagram illustrates a system 100 for the recovery of desired light hydrocarbons from waste gas, in refinery gas plants using a back-end turboexpander.

A raw feed gas, which contains light hydrocarbons, is obtained from a coker or FCC main fractionator overhead drum, or from any other source, such as refinery gas streams, gas streams used for fuel or to be eliminated as waste. The raw feed gas is provided to a gas chiller/dryer 106 via a raw feed gas line 104. In the gas chiller/dryer 106, the raw feed gas is compressed, is amine-treated to remove hydrogen sulfide and carbon dioxide, if necessary, is dried and is chilled, to produce a residue lighter gas. The residue lighter gas is then fed at an outflow through a residue lighter gas line 110 to a distillation column 112. The distillation column 112 includes a distillation column top section 112a, a distillation column bottom section 112c, and a distillation column intermediate section 112d. The distillation column intermediate section 112d is intermediate the distillation column top section 112a and the distillation column bottom section 112c. In the distillation column 112, the desired light hydrocarbons are removed from the residue lighter gas to produce a raw column bottom liquid product, and an overhead product containing methane, and lighter components.

A portion of the raw column bottom liquid product exits the distillation column bottom section 112c through a raw column bottom liquid product line 145a. The system 100 also includes a reboiler 142, which draws another portion of the raw column bottom liquid product from a bottom section 112c of the distillation column 112 through a raw column bottom liquid product line 145b. The reboiler 142 heats and recirculates raw column bottom liquid product to the distillation column bottom section 112c.

The overhead product, a treated gas from which some of the desired light hydrocarbons have been removed, leaves the distillation column 112 at the distillation column top section 112a through an overhead product line 116, and is sent first to a first liquid/gas separator 118, such as a knock out drum, to remove the small volume of liquid potentially present in the overhead product, preventing that liquid from entering the back-end turboexpander 120. The back-end turbo-expander 120 would otherwise freeze any liquid, interfering with the operation of the back-end turboexpander 120. The remaining overhead product is then delivered to the back-end turboexpander 120 where it is further cooled to produce a turboexpander two-phase product containing a condensed liquid, which acts as a reflux for the distillation column 112, and a remaining vapor. The turboexpander two-phase product is transmitted to a second liquid/gas separator 126, such as a knock-out drum, through a turboexpander two-phase product line 124, where the turboexpander two-phase product is separated into the condensed liquid and the remaining vapor. The condensed liquid is sent through a condensed liquid line 132 to a pump 134 and then to the distillation column top section 112a as a reflux.

The remaining vapor is sent through a remaining vapor line 130 to the shell side of a shell and first tube condenser 112b located in the distillation column 112 near the distillation column top section 112a to provide indirect cooling of column vapors within the distillation column 112 and to exit as a residue gas. The first tube condenser 112b increases the efficiency of the distillation column in separating the constituents of the residue gas. The first tube condenser 112b may consist of spaced-apart vertical condenser tubes within a shell of the distillation column 112 where the column internal vapors may flow inside the condenser tubes. The first tube condenser 112b is in communication with the remaining vapor line 130, so that the remaining vapor is fed into the void between the vertical condenser tubes. The remaining vapor from the remaining vapor line 130 has a temperature lower than that of the column internal vapors within the distillation column 112, so that the remaining vapor acts as a cooling medium. While passing through the void between the vertical condenser tubes of the first tube condenser 112b, the remaining vapor absorbs heat from the column internal vapors within the distillation column 112, and exits as heated remaining vapor. Using the remaining vapor, which otherwise would be waste, as a cooling medium in the first tube condenser 112b, maximizes the limited value of the remaining vapor.

In a further embodiment, the distillation column 112 may be a deethanizer.

To further increase the efficiency of a deethanizer distillation column 112, a second condenser 150 may be provided above the distillation column intermediate section 112d. The second condenser 150 permits the heat transfer from the heated contents of the distillation column 112, increasing the rate of distillation and therefore the efficiency of the distillation column 112. A cooled refrigerant 152 is provided to the second condenser 150 and removed as heated refrigerant 154. The second condenser 150 may be external to the distillation column together with piping and pumping to provide material from within the distillation column 112 and back to the distillation column 112. Preferably, the second condenser 150 is internal to and within the distillation column 112, so that external components can be minimized.

The second condenser 150 may consist of spaced-apart vertical tubes within a shell of the distillation column 112 where the column internal vapors may flow inside the condenser tubes. A cooled refrigerant 152, having a temperature lower than the residue gas within the distillation column 112 is fed into the space between the condenser tubes within the distillation column 112 to act as a cooling medium. While passing through the void between the vertical condenser tubes of the second condenser 150, the cooled refrigerant 152 absorbs heat from the column internal vapors within the distillation column 112, and exits as heated refrigerant 154. The heated refrigerant 154 is then compressed, condensed, permitted to expand, and returned to the second condenser 150 as cooler refrigerant 152.

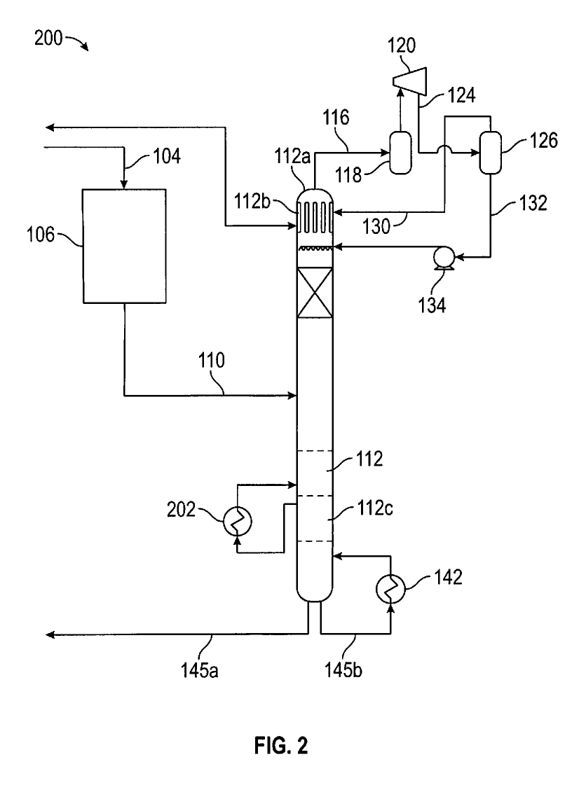

Referring now to FIG. 2, a schematic diagram illustrates another system 200 for the recovery of certain light hydrocarbons from waste gas using a back-end turboexpander and a heat exchanger. The distillation column 112 may be a demethanizer. Consistent with demethanizers, the system 200 further includes a heat exchanger 202 in communication with the distillation column 112 in the distillation column intermediate section 112d. The heat exchange 202 draws a portion of a partially-distilled liquid from the distillation column intermediate section 112d, heats the portion of a partially-distilled liquid to produce a heated portion of a partially-distilled liquid, and provides the heated portion of a partially-distilled liquid to the distillation column 112 in the distillation column intermediate section 112d.

The system 100 may provide a higher recovery of the desired light hydrocarbons, particularly propane and propylene, from the feed to refinery gas plants, as much as 99% compared to the 90-94% recovery in conventional absorber-stripper gas plants. Additionally up to 50% of the ethylene and ethane can be recovered, if recovery of these components is desired. The system 200 may provide an even higher recovery, in the range of 3-5%, of ethylene and ethane from the refinery waste gases compared to the conventional configuration where the turboexpander is positioned between the distillation column and the gas chiller/dryer. Each system also removes the liquid and heavy gas hydrocarbons before entering the turboexpander where they are likely to freeze. Each system disclosed may replace a conventional absorber-stripper design used in refinery gas plants used to recover the desired light hydrocarbons. Each system may also be retrofitted into existing refinery gas plants. A cryogenic gas plant utilizing either system would provide higher recovery of propane and would permit recovery of part of the ethane in the gas plant feed.

While the present disclosure has been described in connection with presently preferred embodiments, it will be understood by those skilled in the art that it is not intended to limit the disclosure to those embodiments. For example, it is anticipated that by routing certain streams differently or by adjusting operating parameters, different optimizations and efficiencies may be obtained, which would nevertheless not cause the system to fall outside of the scope of the present disclosure. It is therefore, contemplated that various alternative embodiments and modifications may be made to the disclosed embodiments without departing from the spirit and scope of the disclosure defined by the appended claims and equivalents thereof.

1. A system for recovery of light hydrocarbons, which comprises:
a gas chiller/dryer;
a distillation column connected to the gas chiller/dryer for recovering the light hydrocarbons;
a first liquid/gas separator connected to the distillation column for separating the light hydrocarbons in a liquid state from the distillation column and the light hydrocarbons in a gas state from the distillation column; and
a turboexpander connected to the first liquid/gas separator for cooling the light hydrocarbons in a gas state to produce a condensed liquid and a remaining vapor.

2. The system of claim 1, wherein the distillation column is a deethanizer.

3. The system of claim 1, wherein the distillation column is a demethanizer.

4. The system of claim 1, further comprising:
a heat exchanger in communication with the light hydrocarbons in the distillation column.

5. The system of claim 1, wherein the light hydrocarbons comprise ethylene and the light hydrocarbons having a molecular weight greater than ethylene.

6. A method for recovering light hydrocarbons from a gas stream, which comprises:
treating the gas stream by compressing, amine-treating, drying and chilling to produce a residue lighter gas; and
separating the residue lighter gas in a distillation column between an overhead product and a raw column bottom liquid product containing the light hydrocarbons.

7. The method of claim 6, further comprising:
heating a portion of the raw column bottom liquid product in a first heat exchanger to obtain a heated portion of the raw column bottom liquid product; and
injecting the heated portion of the raw column bottom liquid product into the distillation column.

8. The method of claim 6, wherein the distillation column is a deethanizer.

9. The method of claim 6, wherein the distillation column is a demethanizer.

10. The method of claim 9, further comprising:
heating a portion of the raw column bottom liquid product in a first heat exchanger to obtain a heated portion of the raw column bottom liquid product; and
injecting the heated portion of the raw column bottom liquid product into the distillation column.

11. The method of claim 10, further comprising:
heating a portion of the raw column bottom liquid product in a second heat exchanger to obtain a second heated portion of the raw column bottom liquid product; and
injecting the second heated portion of the raw column bottom liquid product into the distillation column.
